(19) United States
(12) Patent Application Publication
(10) Pub. No.: US 2019/0021926 A1
(43) Pub. Date: Jan. 24, 2019

(54) PATIENT COOLING SYSTEM RESPONSIVE TO HEAD ELEVATION

(71) Applicant: Hill-Rom Services, Inc., Batesville, IN (US)

(72) Inventors: Charles A. LACHENBRUCH, Batesville, IN (US); Joshua A. WILLIAMS, West Harrison, IN (US)

(21) Appl. No.: 16/027,533

(22) Filed: Jul. 5, 2018

Related U.S. Application Data
(60) Provisional application No. 62/535,324, filed on Jul. 21, 2017.

(51) Int. Cl. A61G 7/057; A61F 7/00; A61G 7/015; A61G 7/018

(52) U.S. Cl. CPC A61G 7/05784; A61F 7/00; A61G 7/015; A61G 2210/70; A61F 2007/006; A61F 2007/0067; A61G 7/018

(57) ABSTRACT

A patient support structure includes a cushion layer. A microclimate structure is integrated atop the cushion layer. The microclimate structure includes an upper layer having a vapor and liquid permeable therapeutic region, an air permeable middle layer, and a liquid impermeable lower layer. A first plurality of perforations extends through the upper layer of the microclimate structure in a seat section of the microclimate structure. A second plurality of perforations extends through the upper layer of the microclimate structure in a head section of the microclimate structure. When the microclimate structure is in a first position, air is supplied through the first plurality of perforations and the second plurality of perforations. When the microclimate structure is in a second position, air is supplied through the first plurality of perforations and air flow through the second plurality of perforations is limited.

### CROSS-REFERENCE TO RELATED PATENT APPLICATION

This application claims priority to U.S. Provisional Patent Application Ser. No. 62/535,324, filed Jul. 21, 2017 and titled “PATIENT COOLING SYSTEM RESPONSIVE TO HEAD ELEVATION,” which is herein incorporated by reference in its entirety.

Cross-reference is made to Patent Cooperation Treaty Patent Application No. PCT/US2016/34908 entitled “PATIENT SUPPORT APPARATUS,” which was filed on May 29, 2016, the entirety of which is incorporated herein by reference.

### BACKGROUND

The present disclosure is related to bed mattresses for supporting patients. More specifically, the present disclosure is related to a microclimate structure for hospital beds, medical beds, or other types of beds in which the microclimate structures are designed to cool and dry a patient's skin around targeted therapeutic regions.

In a care facility, such as a hospital or a nursing home, patients are often placed on patient support apparatuses for an extended period of time. Patients who are positioned on the patient support apparatus often have a risk of developing certain skin condition, such as bed sores (also known as pressure sores or decubitus ulcers), due to heat and moisture along the interface of the patient with the surface of the bed mattress. In an effort to mitigate or prevent such conditions, some bed mattresses have a built-in microclimate structure. The microclimate structure may conduct air along the interface of a patient with the surface to keep the patient's skin cool and dry. Some microclimate structures require a large volume of air to be supplied to them in order to provide an effective amount of cooling and drying to a patient's skin.

### SUMMARY

The present application discloses one or more of the features recited in the appended claims and/or the following features which, alone or in any combination, may comprise patentable subject matter:

According to one aspect of the disclosure, a patient support apparatus includes a frame and an air box. A patient support structure is supported by the frame and includes a head section, a foot section, and a seat section between the head section and foot section. The patient support structure includes a cushion layer and an outer ticking layer including an upper surface portion positioned to support a patient. A microclimate structure is positioned within the outer ticking layer and between the cushion layer and the upper surface portion. The microclimate structure includes an upper layer. At least a portion of the upper layer is vapor and liquid permeable. A middle layer is air permeable. A lower layer is liquid impermeable. A first plurality of perforations extends through the upper layer of the microclimate structure in the seat section of the patient support structure. A second plurality of perforations extends through the upper layer of the microclimate structure in the head section of the patient support structure. When the patient support structure is in a first position, the air box supplies air flow through the first plurality of perforations and the second plurality of perforations. When the patient support structure is in a second position, the air box supplies air flow through the first plurality of perforations and air flow through the second plurality of perforations is limited.

In some embodiments, when in the second position, a crease may be formed in the microclimate structure between the first plurality of perforations and the second plurality of perforations. In some embodiments, the crease formed in the microclimate structure may limit the air flow from the air box to the second plurality of perforations. In some embodiments, when in the first position, the head section may be declined. In some embodiments, when in the second position, the head section may be inclined. In some embodiments, the microclimate structure may extend from a lower end of the head section to a lower end of the seat section of the patient support structure, excluding the foot section of the patient support structure. In some embodiments, the air box may be coupled to a conduit to conduct pressurized air through the microclimate structure.

In some embodiments, the vapor and liquid permeable portion of the upper layer of the microclimate structure may define a therapeutic region. In some embodiments, the therapeutic region of the upper layer of the microclimate structure may have a highly breathable, vapor and liquid permeable material. In some embodiments, a non-therapeutic region of the upper layer of the microclimate structure may have a vapor permeable but liquid impermeable material. In some embodiments, the middle layer of the microclimate structure may have a three-dimensional material configured to conduct air between the upper layer and the lower layer of the microclimate structure. In some embodiments, the middle layer of the microclimate structure may have more than one section of the three dimensional material, in which at least one section of the three dimensional material conducts and delivers air along a therapeutic region.

In some embodiments, the foot section of the microclimate structure may be a foam padding. In some embodiments, the cushion layer may have a first inflatable support bladder and a second inflatable support bladder, and an air distribution sleeve that extends between the first inflatable support bladder and the second inflatable support bladder. In some embodiments, the cushion layer may have foam paddings. In some embodiments, the outer ticking layer may be a vapor permeable and liquid impermeable material. In some embodiments, the outer ticking layer may encase the microclimate structure. In some embodiments, the outer ticking layer may encase the microclimate structure and the cushion layer.

According to another aspect of the disclosure, a patient support structure includes a cushion layer. A microclimate structure is integrated atop the cushion layer. The microclimate structure includes an upper layer having a vapor and liquid permeable therapeutic region, an air permeable middle layer, and a liquid impermeable lower layer. A first plurality of perforations extends through the upper layer of the microclimate structure in a seat section of the microclimate structure. A second plurality of perforations extends through the upper layer of the microclimate structure in a head section of the microclimate structure. When the microclimate structure is in a first position, air is supplied through the first plurality of perforations and the second plurality of perforations. When the microclimate structure is in a second position, air is supplied through the first plurality of perforations and air flow through the second plurality of perforations is limited.

In some embodiments, when in the second position, a crease may be formed in the microclimate structure between the first plurality of perforations and the second plurality of perforations. In some embodiments, the crease formed in the microclimate structure may limit the air flow to the second plurality of perforations. In some embodiments, when in the first position, the head section may be declined. In some embodiments, when in the second position, the head section may be inclined.

In some embodiments, a therapeutic region of the microclimate structure may include a highly breathable, vapor and liquid permeable material. In some embodiments, the middle layer of the microclimate structure may include a three-dimensional material configured to conduct air between the upper layer and the lower layer of the microclimate structure. In some embodiments, the middle layer of the microclimate structure may include more than one section of the three dimensional material. At least one section of the three dimensional material may conduct and deliver air along a therapeutic region.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a perspective view from a foot end on the patient's right of a patient support apparatus including an air box and a patient support structure supported on a frame;

FIG. 2 is a top plan view of the patient support apparatus of FIG. 1 where a targeted therapeutic region extends from a head section through a seat section of the patient support structure, covering an entire upper surface of the microclimate structure;

FIG. 3 is a cross section taken along section lines 3-3 of FIG. 2 showing an embodiment of the patient support structure encased by an outer ticking layer where an upper ticking covers the microclimate structure and a lower ticking encases a cushion layer;

FIG. 4 is a perspective view of the patient support structure having a head end in an inclined position; and

FIG. 5 is a side elevation view of the patient support structure having a head end in an inclined position.

### DETAILED DESCRIPTION

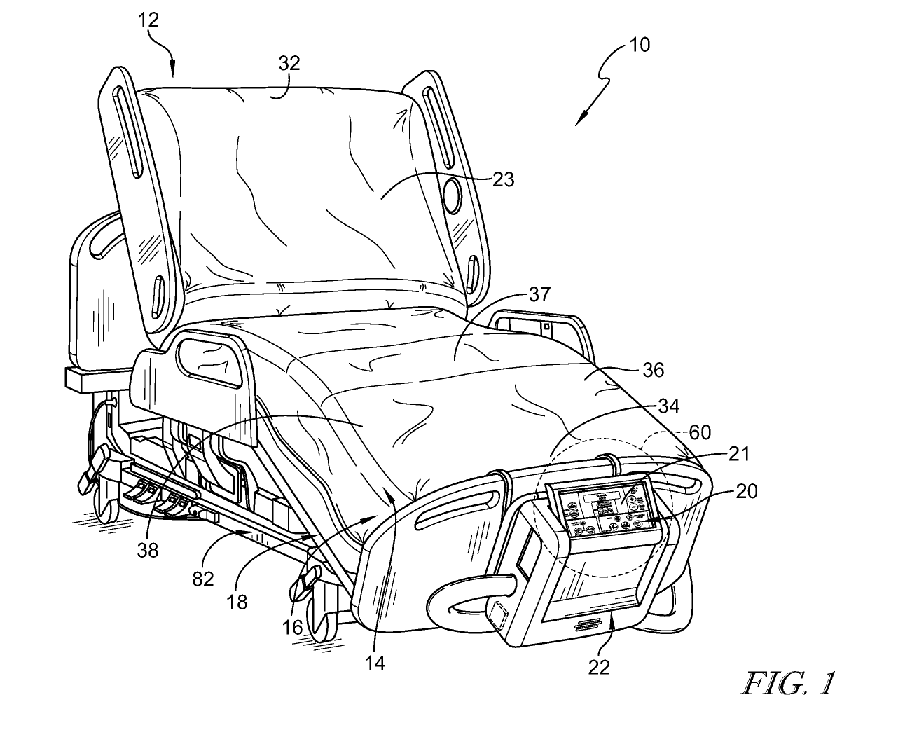

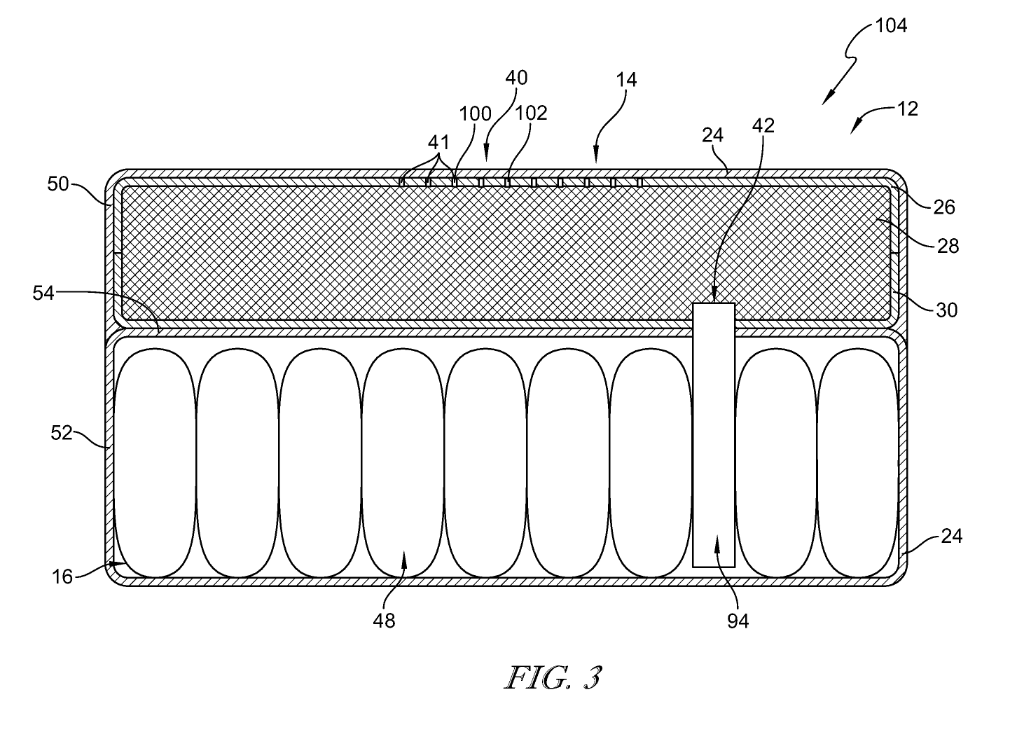

An illustrative patient support apparatus 10 embodied as a hospital bed is shown in FIG. 1. The patient support apparatus 10 includes a frame 18, a patient support structure 12 supported on the frame 18, and an air box 22. The patient support structure 12 is adapted to support a patient lying on the patient support apparatus 10 and includes a head section 32, a seat section 37, and a foot section 34. As will be discussed in further detail below, the patient support structure 12 further includes a microclimate structure 14 and a cushion layer 16 which supports the microclimate structure 14 as shown in FIG. 3. The cushion layer 16 may include a plurality of inflatable support bladders 48. The microclimate structure 14 is positioned on the cushion layer 16 on an occupant side and adjacent a support surface 23 and is configured to conduct air adjacent the support surface 23 of the patient support structure 12. The air conducted by the microclimate structure 14 is pressurized and pushed through the microclimate structure 14 by the air box 22. By conducting air along an interface of the support surface 23 and the patient, the microclimate structure 14 transfers heat and moisture from the patient and cools and dries the patient's skin in order to reduce the risk of bed sore formation by the patient.

The air box 22 further includes a user interface 60 that is configured to receive user inputs. The user interface 60 includes a display screen 21 and a plurality of buttons 20 for inputting patient information and for controlling operation of the air box 22 and the support surface 23. Particularly, the user interface 60 allows a user to adjust the flow of air provided by the air box 22 to the microclimate structure 14 and, in some embodiments, to adjust the temperature of air provided by the air box 22 to the microclimate structure 14. Specifically, in some embodiments, the user interface 60 may include a patient information input panel, an alarm panel, a lateral rotation therapy panel, an inflation mode panel, a normal inflation control panel, and a microclimate control panel.

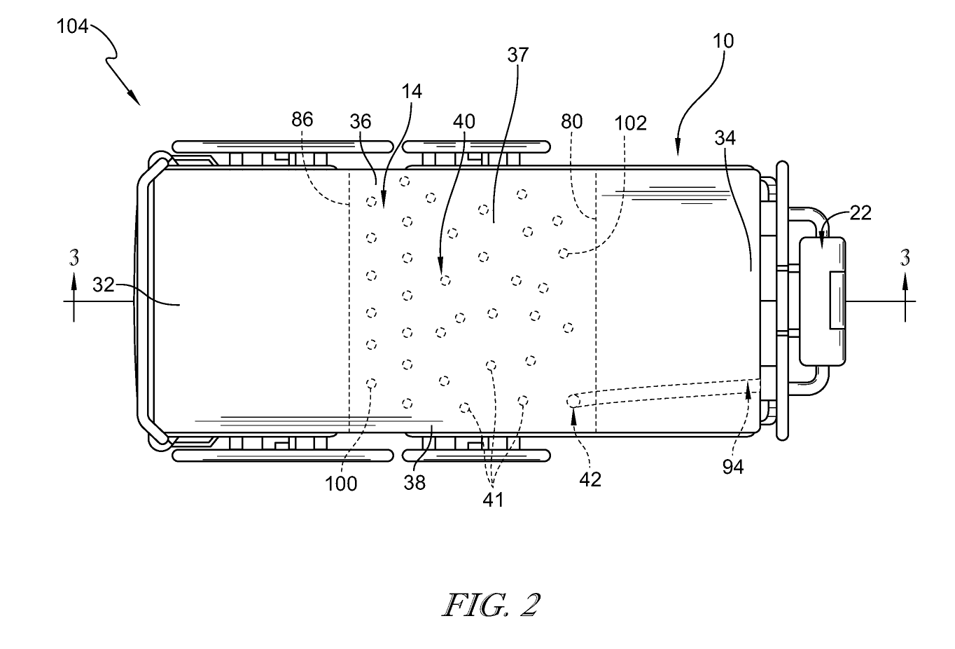

The microclimate structure 14 is configured to receive pressurized air from the air box 22 and to conduct air through the microclimate structure 14 to cool and dry the interface between a patient and the patient support apparatus 10 to promote skin health by removing patient heat and moisture along the interface when the patient is supported on the patient support apparatus 10. The microclimate structure 14 generally spans laterally from a left side 36 to a right side 38 and extends longitudinally from above a lower end 86 of the head section 32 to a lower end 80 of the seat section 37, excluding the foot section 34 of the patient support structure 12 as shown in FIG. 2.

Referring to FIG. 2, in one embodiment, the microclimate structure 14 further includes a therapeutic region 40 which is specifically configured to target specific areas of the patient's body where local climate control is most needed. This corresponds to the areas where the pressure of patient's weight against the support surface 23 is the greatest when the patient is lying supine and centered on the microclimate structure 14. The therapeutic region 40 may be made from a highly breathable material or a perforated material. Because the patient's sweat glands are distributed non-uniformly throughout the patient's body, perspiration tends to accumulate on the skin of the patient's torso and pelvic region. Therefore, the shape of the therapeutic region 40 is designed to provide a local climate control to those areas that are generally prone to moisture accumulation. The therapeutic region 40 is in the head section 32 and seat section 37 of the patient support structure 12 as shown in FIG. 2. The large therapeutic region 40 ensures to underlie the patient's torso and pelvic region. By reducing the area of the therapeutic region 40 through which the air box 22 is required to push air, the microclimate structure 14 allows for reduction of the pressure and flow needed from an air source included in the air box 22.

The microclimate structure 14 includes a fluid flowpath having an inlet port 42. The fluid flowpath spans laterally across the microclimate structure 14 from its right side 38 to its left side 36 and extends longitudinally through the microclimate structure 14 to the head section 32 of the patient support structures 12. The inlet port 42 is directly coupled to the air box 22 via a distribution sleeve 94 and is located at the lower end 80 of the seat section 37 of the patient support structure 12. Thus, air from the air box 22 is introduced into the microclimate structure 14 at the origination point or inlet port 42 near the pelvic region of the patient lying on the microclimate structure 14. By directing the location of air introduction from the air box 22 closer to the therapeutic region 40, the microclimate structure 14 will provide an effective amount of cooling and drying to a patient's skin at the specific targeted areas, and achieve the effective result with minimal air. Having the inlet port 42 near the therapeutic region 40 prevents air from diffusing out of the microclimate structure 14 while the air flows from the inlet port 42 to the therapeutic region 40, thus requiring less volume of air. However, in some embodiments, the inlet port 42 may be positioned at the foot end of the microclimate structure 14. Further, the microclimate structure 14 may have an outlet at the head section 32 of the patient support structure 12 to exhaust the air and/or liquid. Other inlet port and outlet designs may be used in other embodiments. When the outlet is omitted, the air that traverses the microclimate structure 14 is pushed out through the perforations 41 in the therapeutic region 40 and escapes through an outer ticking layer 24 of the patient support 12. The perforations 41 include head section perforations 100 and seat section perforations 102.

An outer ticking layer 24 encompasses the microclimate structure 14 as shown in FIG. 3. The outer ticking layer 24 includes an upper ticking layer 50 and a lower ticking layer 52. The upper ticking layer 50 covers the microclimate structure 14 and the lower ticking layer 52 encases the cushion layer 16. The upper ticking layer 50 includes a breathable material that is vapor permeable but liquid impermeable. This allows the patient heat and moisture to flow away from the patient's skin in form of vapor and pass through the upper ticking layer 50 into the area which encloses the microclimate structure 14. The vapor then condenses between the upper ticking layer 50 and a first or upper layer 26 of the microclimate structure 14. At least a portion of the upper layer 26 includes a vapor and liquid permeable material which defines the therapeutic region 40. In the illustrative embodiment, the therapeutic region 40 of the upper layer 26 includes a number of perforations 41 that allows the condensed moisture and liquid from the therapeutic region 40 to flow through the upper layer 26 into a middle layer 28 of the microclimate structure 14. The upper layer 26 includes a vapor permeable but liquid impermeable material to allow vapor to flow through the upper layer 26. In the illustrated embodiments, the microclimate structure 14 and the cushion layer 16 are separated by a middle ticking layer 54, which is a top layer of the lower ticking layer 52. However, in some embodiments, a unitary outer ticking layer 24 may encase the entire patient support structure 12, including the microclimate structure 14 and the cushion layer 16.

The material of the middle layer 28 is a three-dimensional material. The three-dimensional material is arranged to extend from the upper end of the head section 32 to the lower end of the foot section 34 of the patient support structure 12 as shown in FIG. 3. The three-dimensional material is air and liquid permeable. The inlet port 42 is coupled to the lower end 80 of the seat section 37 of the three-dimensional material to allow air from the air box 22 to flow between the upper layer 26 and a lower layer 30 of the microclimate structure 14 and from the lower end 80 of the seat section 37 to the head section 32 of the patient support structure 12. Therefore, once the moisture and liquid reach the middle layer 28 from the upper layer 26, the moisture and liquid are carried away and evaporated by air flowing through the middle layer 28. The cooled-vapor can then be either directed toward the outlet or back toward the support surface 23 to cool and dry the patient's skin around the interface of the patient's skin with the support surface 23. The cooled-vapor directed toward the support surface 23 passes through the head section perforations 100 and the seat section perforations 102.

Lastly, the lower layer 30 of the microclimate structure 14 includes a liquid impermeable material to prevent liquid from leaking through the lower layer 30 into the cushion layer 16. Illustratively, the cushion layer 16 includes the inflatable support bladders 48 to support the microclimate structure 14 as shown in FIG. 3. Accordingly, the air box 22 is coupled to the microclimate structure 14 and the inflatable support bladders 48 to provide pressurized air to the support surface 23 and the cushion layer 16. In other embodiments, the cushion layer 16 may omit some or all of the inflatable support bladders 48 and utilize foam cushioning structures instead of the inflatable support bladders 48.

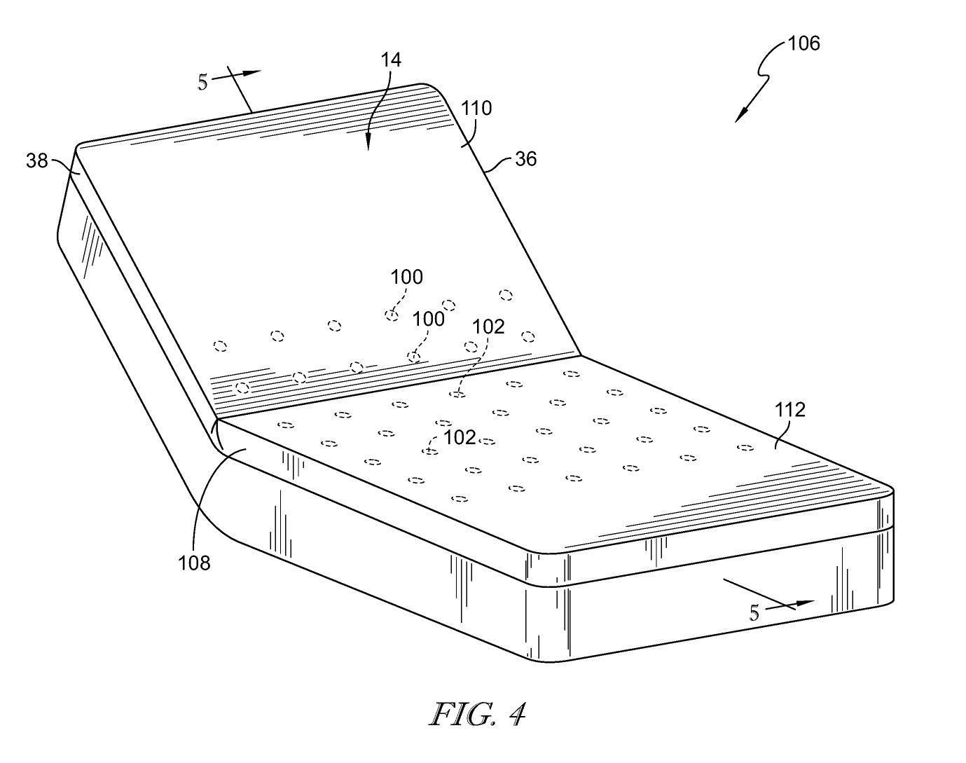

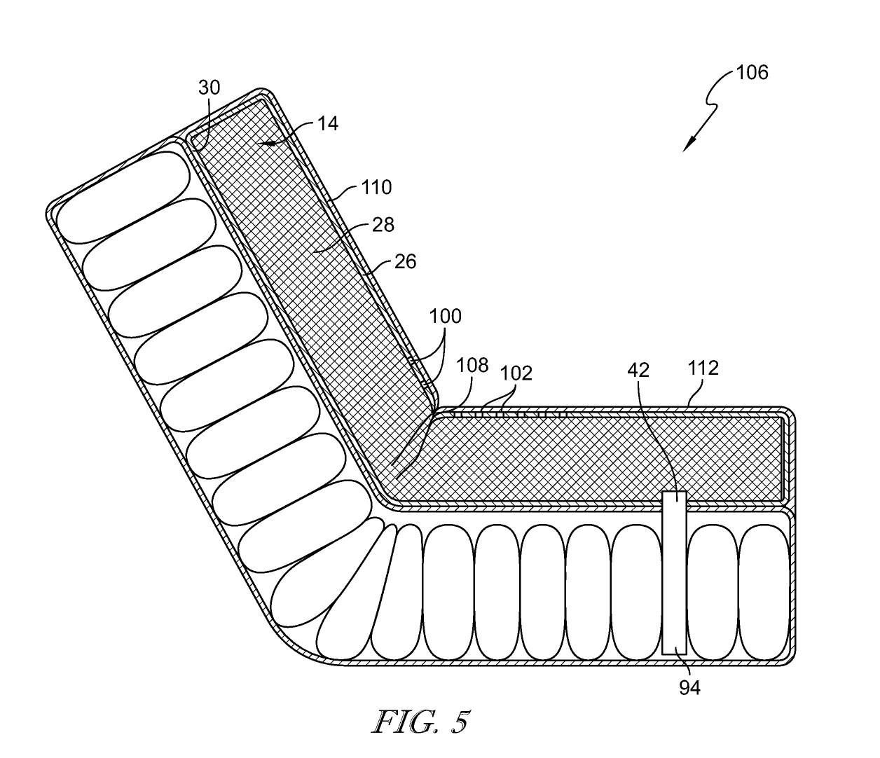

The patient support apparatus 10 is moveable between a declined position 104, shown in FIGS. 2 and 3, and an inclined position 106, shown in FIGS. 4 and 5. The patient support apparatus 10 moves between the declined position 104 and the inclined position 106 by altering an angle of the head section 32 relative to the seat section 37. In the declined position 104, the head section 32 extends substantially planar with the seat section 37 so that a patient may lie supine on the patient support structure 12. In the inclined position 106, the head section 32 is angled relative to the seat section 37 so that the patient may sit up in the patient support structure 12, thereby applying pressure to the seat section 37. When the patient support apparatus 10 is moved between the declined position 104 and the inclined position 106, the patient support structure 12 and the microclimate structure 14 also move between a declined and inclined position. When moved to the inclined position, the inflatable support bladders 48 may maintain an air pressure therein. Alternatively or in addition to, at least some of the inflatable support bladders 48 may deflate or inflate to accommodate the change in position of the patient support structure 12 between the declined position and the inclined position.

In the declined position 104, air flows from the distribution sleeve 94 into the microclimate structure 14 through inlet 42. The moisture and liquid in the middle layer 28 are carried away and evaporated by the air flowing through the middle layer 28. The cooled-vapor can then be either directed toward the outlet or back toward the support surface 23 to cool and dry the patient's skin around the interface of the patient's skin with the support surface 23. The cooled-vapor directed toward the support surface 23 passes through both the head section perforations 100 and the seat section perforations 102.

Referring to FIGS. 4 and 5, when in the inclined position 106, a crease 108 is formed in the microclimate structure 14. The crease 108 is formed between a head section 110 of the microclimate structure 14 and a seat section 112 of the microclimate structure 14. The crease 108 spans laterally across the microclimate structure 14 from its right side 38 to its left side 36. The crease 108 is positioned between the head section perforations 100 and the seat section perforations 102. The crease 108 extends from the upper layer 26 of the microclimate structure 14 to the lower layer 30 of the microclimate structure 14 through the middle layer 28. The crease 108 limits the three-dimensional space within the middle layer 28.

The middle layer 28 may have additional material properties in the location of the crease 108. Such additional material properties facilitate forming the crease 108 and preventing airflow through the middle layer 28. In some embodiments, the middle layer 28 includes a crease material, e.g. a rubber material, a plastic material, a foam, etc., in the location where the crease 108 is formed. Alternatively, or in addition to, the middle layer 28 may have a higher density in the location where the crease 108 is formed. The additional and/or thicker material facilitates forming the crease 108 to pinch the middle layer 28, thereby limiting the airflow through the crease 108. In some embodiments, the middle layer 28 has less material or a lower density at the location where the crease 108 is formed. The reduction in material allows the ticking layer around the middle layer 28 to fold over, thereby creating the crease 108.

In some embodiments, the crease 108 extends entirely from the upper layer 26 to the lower layer 28. In such an embodiment, air flow is substantially prevented from passing through the crease 108. That is, air flow from the distribution sleeve 94 passes from inlet 42 into the seat section 112 of the microclimate structure 14, but is substantially prevented from entering the head section 110 of the microclimate structure 14. As such, the cooled-vapor formed in the middle section 28 of the microclimate structure 14 is directed toward the support surface 23 and passes through the seat section perforations 102, while being substantially prevented from passing through the head section perforations 100.

In some embodiments, the crease 108 extends partially from the upper layer 26 to the lower layer 28. In such an embodiment, air flow is limited from passing through the crease 108. The amount that air flow is limited may be determined by a size of the crease 108, i.e. how far the crease 108 extends from the upper layer 26 to the lower layer 28. Air flow from the distribution sleeve 94 passes into a seat section 112 of the microclimate structure 14 and is limited from entering the head section 110 of the microclimate structure 14, i.e. air flow into the head section 110 is starved. As such, the cooled-vapor formed in the middle section 28 of the microclimate structure 14 is directed toward the support surface 23 and passes through the seat section perforations 102, while being limited from passing through the head section perforations 100.

Although this disclosure refers to specific embodiments, it will be understood by those skilled in the art that various changes in form and detail may be made without departing from the subject matter set forth in the accompanying claims.

1. A patient support apparatus comprising:
a frame,
an air box, and
a patient support structure being supported by the frame and including a head section, a foot section, and a seat section between the head section and foot section, the patient support structure further comprising:
a cushion layer,
an outer ticking layer including an upper surface portion positioned to support a patient,
a microclimate structure positioned within the outer ticking layer and between the cushion layer and the upper surface portion, the microclimate structure comprising an upper layer, at least a portion of the upper layer being vapor and liquid permeable, a middle layer being air permeable, and a lower layer being liquid impermeable,
a first plurality of perforations extending through the upper layer of the microclimate structure in the seat section of the patient support structure, and
a second plurality of perforations extending through the upper layer of the microclimate structure in the head section of the patient support structure,
wherein, when the patient support structure is in a first position, the air box supplies air flow through the first plurality of perforations and the second plurality of perforations, and, when the patient support structure is in a second position, the air box supplies air flow through the first plurality of perforations and air flow through the second plurality of perforations is limited.

2. The patient support apparatus of claim 1, wherein, when in the second position, a crease is formed in the microclimate structure between the first plurality of perforations and the second plurality of perforations.

3. The patient support apparatus of claim 2, wherein the crease formed in the microclimate structure limits the air flow from the air box to the second plurality of perforations.

4. The patient support apparatus of claim 1, wherein, when in the first position, the head section is declined.

5. The patient support apparatus of claim 4, wherein, when in the second position, the head section is inclined.

6. The patient support apparatus of claim 1, wherein the microclimate structure extends from a lower end of the head section to a lower end of the seat section of the patient support structure, excluding the foot section of the patient support structure.

7. The patient support apparatus of claim 1, wherein the air box is further coupled to a conduit to conduct pressurized air through the microclimate structure.

8. The patient support apparatus of claim 1, wherein the vapor and liquid permeable portion of the upper layer of the microclimate structure defines a therapeutic region.

9. The patient support apparatus of claim 8, wherein the therapeutic region of the upper layer of the microclimate structure comprises a highly breathable, vapor and liquid permeable material.

10. The patient support apparatus of claim 8, wherein a non-therapeutic region of the upper layer of the microclimate structure comprises a vapor permeable but liquid impermeable material.

11. The patient support apparatus of claim 1, wherein the middle layer of the microclimate structure comprises a three-dimensional material configured to conduct air between the upper layer and the lower layer of the microclimate structure.

12. The patient support apparatus of claim 1, wherein the middle layer of the microclimate structure comprises more than one section of the three dimensional material, in which at least one section of the three dimensional material conducts and delivers air along a therapeutic region.

13. The patient support apparatus of claim 1, wherein the foot section of the microclimate structure comprises a foam padding.

14. The patient support apparatus of claim 1, wherein the cushion layer includes a first inflatable support bladder and a second inflatable support bladder, and an air distribution sleeve extends between the first inflatable support bladder and the second inflatable support bladder.

15. The patient support apparatus of claim 1, wherein the cushion layer includes foam paddings.

16. The patient support apparatus of claim 1, wherein the outer ticking layer comprises a vapor permeable and liquid impermeable material.

17. The patient support apparatus of claim 1, wherein the outer ticking layer encases the microclimate structure.

18. The patient support apparatus of claim 1, wherein the outer ticking layer encases the microclimate structure and the cushion layer.

19. The patient support apparatus of claim 1, further comprising a crease material in the middle layer where the crease is formed.

20. The patient support apparatus of claim 19, wherein the crease material comprises at least one of a rubber material, a plastic material, or a foam.

21. The patient support apparatus of claim 1, wherein the middle layer has a higher density where the crease is formed.

22. The patient support apparatus of claim 1, wherein the middle layer has a lower density where the crease is formed.
